(19) United States
(12) Patent Application Publication
(10) Pub. No.: US 2019/0256498 A1
(43) Pub. Date: Aug. 22, 2019

(54) CRYSTALLINE FORMS OF DACLATASVIR DIHYDROCHLORIDE

(71) Applicant: LUPIN LIMITED, Pune, Maharashtra (IN)

(72) Inventors: Palash SANPHUI, Pune, Maharashtra (IN); Radhakrishna Bhikaji SHIVDAVKAR, Pune, Maharashtra (IN); Gurvinder Pal SINGH, Pune, Maharashtra (IN); Purna Chandra RAY, Pune, Maharashtra (IN); Girij Pal SINGH, Pune, Maharashtra (IN); Vikas Ashokrao SADAPHAL, Pune, Maharashtra (IN); Lalitkumar Dilipising RAJPUT, Pune, Maharashtra (IN); Hemraj Mahadeorao LANDE, Pune, Maharashtra (IN)

(21) Appl. No.: 16/316,213

(22) PCT Filed: Jul. 7, 2017
(86) PCT No.: PCT/IB2017/054092

§ 371 Date: Jan. 8, 2019

(30) Foreign Application Priority Data
Jul. 8, 2016 (IN) 201621023521

(51) Int. Cl. C07D 403/14

(52) U.S. Cl. CPC C07D 403/14; C07B 2200/13

(57) ABSTRACT

The present invention provides novel crystalline form L1 of daclatasvir dihydrochloride wherein the diffraction peaks at 5.35, 6.04, 6.76, 8.65, 9.21, 10.35, 10.32, 10.75, 11.04, 11.66, 12.95, 13.56, 16.06, 18.11, 20.58, 22.18, 23.27±0.2 degree two theta in an X-ray diffraction pattern. The present invention provides novel crystalline form L2 of daclatasvir dihydrochloride wherein the diffraction peaks at 8.27, 9.54, 11.06, 13.61, 15.34, 19.01, 22.26, 23.24, 24.73, 25.35±0.2 degree two theta in an X-ray diffraction pattern. The present invention further provides process for preparation of crystalline forms L1 and L2 of daclatasvir dihydrochloride.

### FIELD OF THE INVENTION

The present invention relates to novel crystalline forms of daclatasvir dihydrochloride and process for their preparation.

### BACKGROUND OF THE INVENTION

Daclatasvir is an inhibitor of HCV nonstructural protein 5A (NS5A). It is used for the treatment of patients with chronic hepatitis C virus (HCV) genotype 3 infection. It is developed and marketed by Bristol-Myers Squibb under the trade name Daklinza®. It is on the World Health Organization's List of Essential Medicines. The chemical name for drug substance daclatasvir dihydrochloride is N,N′-[[1,1′-biphenyl]-4,4′-diylbis[1H-imidazole-5,2-diyl-(2S)-2,1-pyrrolidinediyl[(1S)-1-(1-methylethyl)-2-oxo-2,1-ethanediyl]]]bis-carbamic acid, C,C′-dimethyl ester, hydrochloride (1:2). Daclatasvir dihydrochloride (I) has the following structural formula:

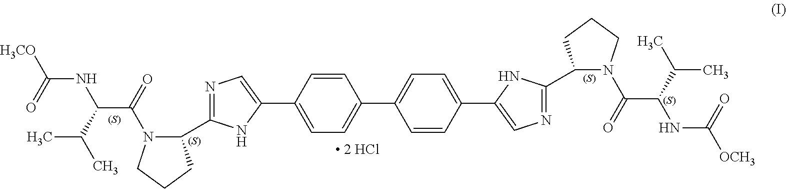

Daclatasvir and its pharmaceutically acceptable salts are described in the PCT application WO 2008021927. Polymorphism has been observed for daclatasvir dihydrochloride, the European medical agency assessment report for Daklinza mentions that two neat crystalline dihydrochloride salts, N1 and N2 have been identified in screening studies and that form N-2 is the thermodynamically most stable polymorph. The crystalline form N-2 of daclatasvir dihydrochloride is discussed in detail in the PCT application WO 2009020828. Other PCT applications WO 2016075588 and WO 2016102979 describe amorphous form of daclatasvir dihydrochloride.

The present invention provides novel crystalline forms of daclatasvir dihydrochloride.

### SUMMARY OF THE INVENTION

The present invention provides novel crystalline form L1 of daclatasvir dihydrochloride characterized by diffraction peaks at 5.35, 6.04, 6.76, 8.65, 9.21, 10.35, 10.32, 10.75, 11.04, 11.66, 12.95, 13.56, 16.06, 18.11, 20.58, 22.18, 23.27±0.2 degree two theta in an X-ray diffraction pattern. The present invention also provides novel crystalline form L2 of daclatasvir dihydrochloride characterized by diffraction peaks at 8.27, 9.54, 11.06, 13.61, 15.34, 19.01, 22.26, 23.24, 24.73, 25.35±0.2 degree two theta in an X-ray diffraction pattern. The present invention further provides process for preparation of crystalline forms L1 and L2 of daclatasvir dihydrochloride.

### DESCRIPTION OF DRAWINGS

FIG. 1—X-ray powder diffraction pattern of crystalline form L1 of daclatasvir dihydrochloride.

FIG. 2—X-ray powder diffraction pattern of crystalline form L2 of daclatasvir dihydrochloride.

### DETAILED DESCRIPTION OF THE INVENTION

The present invention relates to novel crystalline forms of daclatasvir dihydrochloride and process for their preparation.

In one embodiment, the present invention provides crystalline form L1 of daclatasvir dihydrochloride.

The crystalline form L1 of daclatasvir dihydrochloride having characteristic diffraction peaks at 5.35, 6.04, 6.76, 8.65, 9.21, 10.35, 10.32, 10.75, 11.04, 11.66, 12.95, 13.56, 16.06, 18.11, 20.58, 22.18, 23.27±0.2 degree two theta in an X-ray diffraction pattern.

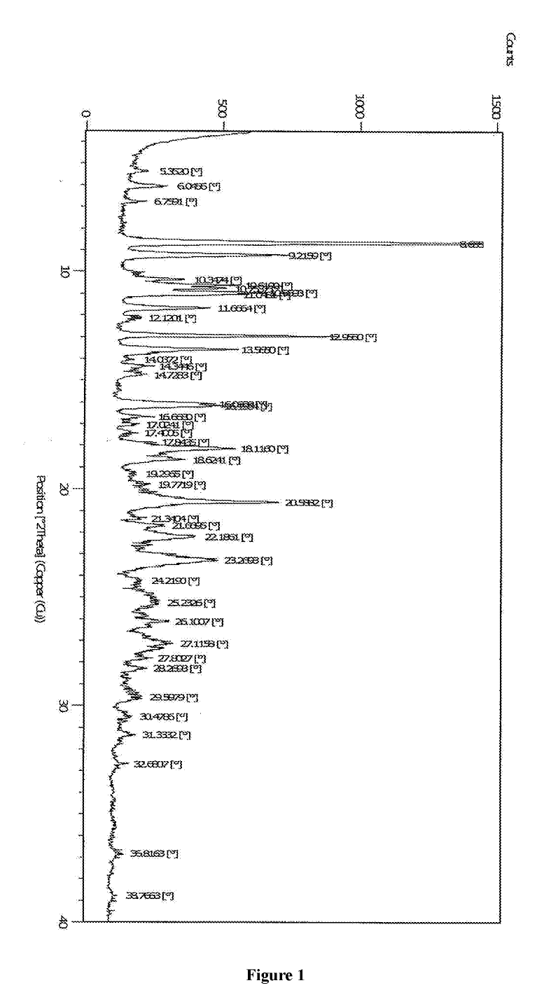

The crystalline form L1 of daclatasvir dihydrochloride of the present invention is characterized by X-ray powder diffraction pattern as depicted in FIG. 1.

The crystalline form L1 of daclatasvir dihydrochloride having characteristic infrared absorption at 3401, 3333, 2963, 2873, 2701, 1718, 1639, 1531, 1443, 1424, 1354, 1271, 1239, 1099, 1029, 827±2 cm⁻¹.

The crystalline form L1 of daclatasvir dihydrochloride is substantially free of other polymorphs of daclatasvir dihydrochloride. The term “substantially free” of other polymorphs means that crystalline form L1 of daclatasvir dihydrochloride does not contain any detectable levels of any other polymorphs of daclatasvir dihydrochloride.

In another embodiment, the present invention provides crystalline form L2 of daclatasvir dihydrochloride.

The crystalline form L2 of daclatasvir dihydrochloride having characteristic diffraction peaks at 8.27, 9.54, 11.06, 13.61, 15.34, 19.01, 22.26, 23.24, 24.73, 25.35±0.2 degree two theta in an X-ray diffraction pattern.

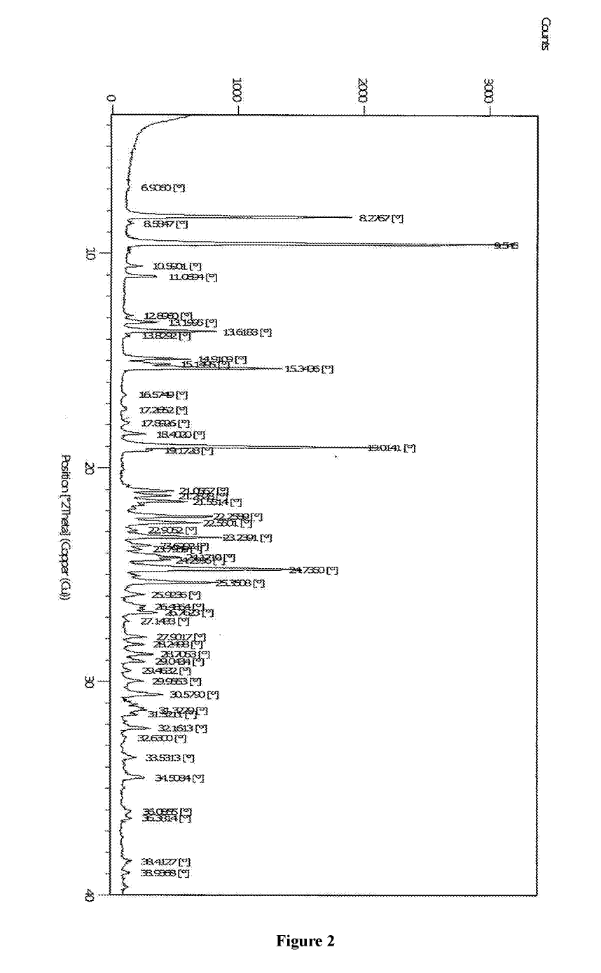

The crystalline form L2 of daclatasvir dihydrochloride of the present invention is characterized by X-ray powder diffraction pattern as depicted in FIG. 2.

The crystalline form L2 of daclatasvir dihydrochloride having characteristic infrared absorption at 3591, 3420, 3217, 1724, 1626, 1509, 1449, 1271, 1230, 1194, 1033, 906, 818, 741±2 cm⁻¹.

The PXRD pattern of crystalline form L2 of daclatasvir dihydrochloride was indexed using the NTREOR code in the program EXP02014 yielding orthorhombic unit cells. Given the volume of unit cell and consideration of density, the number of formula units in the unit cell turned out as Z=4. The unit cell parameters are as mentioned below:

| Crystal System | Orthorhombic |
|---|---|
| a (A°) | 15.9351(8) |
| b (A°) | 21.2896(13) |
| c (A°) | 13.3774(8) |

The crystalline form L2 of daclatasvir dihydrochloride is substantially free of other polymorphs of daclatasvir dihydrochloride. The term “substantially free” of other polymorphs means that crystalline form L2 of daclatasvir dihydrochloride does not contain any detectable levels of any other polymorphs of daclatasvir dihydrochloride.

In yet another embodiment, the present invention provides a process for the preparation of crystalline form L1 of daclatasvir dihydrochloride comprising the steps of:
a) dissolving daclatasvir dihydrochloride in a first solvent,
b) adding the solution of step (a) to a pre cooled solution of second solvent, and
c) isolating form L1 of daclatasvir dihydrochloride.

The first solvent and second solvent can be selected from polar solvent, non-polar solvents or mixtures thereof. Polar solvent can be selected from water, alcohols like methanol, ethanol, butanol, propanol; nitriles like acetonitrile, propionitrile, butyronitrile; ethers like tetrahydrofuran, dioxane, dimethoxyethane; esters like ethyl acetate, ethyl acetoacetate, butyl acetate, propyl acetate; ketones like acetone, methyl ethyl ketone, methyl isobutyl ketone; other polar solvents like dimethylformamide, dimethyl sulfoxide, water and mixtures thereof. Non-polar solvents can be selected from hydrocarbon like hexane, cyclohexane, n-heptane, pentane, cyclopentane, toluene; chlorinated solvents methylene chloride, ethylene chloride, chloroform, carbon tetrachloride and mixtures thereof. The first solvent is preferably dimethyl sulfoxide and second solvent is preferably methanol.

Process for the preparation of crystalline form L1 of daclatasvir dihydrochloride comprises dissolving daclatasvir dihydrochloride in the first solvent by heating the mixture to a temperature of 40° C. to the reflux temperature of the solvent, adding this solution to a second solvent at a temperature of 0 to 30° C. and isolating by techniques known in art like filtration, concentration, removal of solvent by evaporation, distillation, crash cooling, flash evaporation, drying on rotavapor.

In a further embodiment, the present invention provides a process for the preparation of crystalline form L2 of daclatasvir dihydrochloride comprising the steps of:
a) dissolving daclatasvir dihydrochloride in the solvent,
b) stirring the mixture,
c) isolating crystalline form L2 of daclatasvir dihydrochloride.

In the process, daclatasvir dihydrochloride can be used directly or can be prepared in situ by treating daclatasvir with hydrochloric acid in the solvent.

Solvent can be selected from polar solvent, non-polar solvents or mixtures thereof. Polar solvent can be selected from water, alcohols like methanol, ethanol, butanol, propanol; nitriles like acetonitrile, propionitrile, butyronitrile; ethers like tetrahydrofuran, dioxane, dimethoxyethane; esters like ethyl acetate, ethyl acetoacetate, butyl acetate, propyl acetate; ketones like acetone, methyl ethyl ketone, methyl isobutyl ketone; other polar solvents like dimethylformamide, dimethyl sulfoxide, water and mixtures thereof. Non-polar solvents can be selected from hydrocarbon like hexane, cyclohexane, n-heptane, pentane, cyclopentane, toluene; chlorinated solvents methylene chloride, ethylene chloride, chloroform, carbon tetrachloride and mixtures thereof.

The mixture of daclatasvir dihydrochloride, organic solvent and water can be stirred at a temperature of 25° C. to reflux temperature of the solvent for a period of 2 to 48 hours and then can be stirred at a temperature of 0 to 30° C. for a period of 2 to 48 hours and crystalline form L2 of daclatasvir dihydrochloride can be isolated by techniques known in art like filtration, concentration, removal of solvent by evaporation, distillation, crash cooling, flash evaporation, drying on rotavapor.

Daclatasvir dihydrochloride which is used for preparation of crystalline forms L1 and L2 of daclatasvir dihydrochloride of the present invention can be prepared by methods as described in PCT applications WO 2008021927 and WO 2009020828 or by preparations known in the literature.

It has been observed that crystalline forms L1 and L2 of daclatasvir dihydrochloride are stable and do not get converted to any other polymorphic form over a period of time. The crystalline form L2 of daclatasvir dihydrochloride was found to be stable at 5±3° C., 25±2° C./60±5% RH and 40±2° C./75±5% RH. The HPLC purity of crystalline form L2 of daclatasvir dihydrochloride was found to be in the range of 99.6% to 99.8% after 3 months of stability in the above mentioned stability parameters.

Crystalline forms L1 and L2 of daclatasvir dihydrochloride are suitable for preparation of pharmaceutical composition such as tablets and capsules. The pharmaceutical composition containing crystalline forms L1 and L2 of daclatasvir dihydrochloride as the active ingredient along with pharmaceutically acceptable carriers, excipients or diluents can be prepared by methods known in the art. The pharmaceutical compositions containing crystalline forms L1 and L2 of daclatasvir dihydrochloride can be used for treatment of chronic HCV genotype 1 or 3 infection.

The X-ray powder diffraction pattern was recorded at room temperature using PANalytical X′Pert PRO diffractogram with Cu Kα radiation (λ=1.54060 Å), running at 45 kV and 40 mA.

The infrared absorption spectrum was obtained using a Perkin Elmer Precisely Spectrum 400 instrument using KBr pellet method.

The present invention is further illustrated by the following representative examples and does not limit the scope of the invention.

### Examples

### 1. Process for Preparation of Crystalline Form L1 of Daclatasvir Dihydrochloride.

A mixture of daclatasvir dihydrochloride (2 g) and methanol (10 ml) was heated to 60° C. and filtered. The filtrate was added drop-wise to precooled tetrahydrofuran (60 ml) at 10° C. and stirred for 90 minutes. Tetrahydrofuran (40 ml) was added and the mixture was stirred for 16 hours at 25° C. The solid was filtered and dried under vacuum.

### 2. Process for Preparation of Crystalline Form L2 of Daclatasvir Dihydrochloride.

A mixture of daclatasvir dihydrochloride (4.1 g) and ethyl acetate (84 ml) was heated to 70° C. and water (6 ml) was added to it. The mixture was stirred for 10 minutes at 70° C. and then cooled to 25° C. The mixture was again heated to 70° C. and then cooled to 25° C. and stirred for 14 hours. The mixture was heated to 70° C. and ethyl acetate (84 ml) was added to the mixture and then cooled to 25° C. The mixture was stirred for 30 minutes at 25° C. The solid was filtered and dried under vacuum.

### 3. Process for Preparation of Crystalline Form L2 of Daclatasvir Dihydrochloride.

A mixture of daclatasvir dihydrochloride (5 g) and water (20 ml) was heated to 70° C. and stirred for 15 minutes and then cooled to 25° C. Seed crystals were added to it and the mixture was again heated to 50° C. and then cooled to 25° C. The solid was filtered and dried under vacuum.

### 4. Process for Preparation of Crystalline Form L2 of Daclatasvir Dihydrochloride.

A mixture of daclatasvir (5 g) and water (20 ml) was stirred for 15 minutes followed by addition of concentrated hydrochloric acid (1.5 ml). The mixture was heated to 45° C. and stirred for 10 minutes and then cooled to 25° C. The mixture was filtered and the filtrate was collected. Seed crystals were added to the filtrate and the mixture was again heated to 45° C. and stirred for 10 minutes and then cooled to 25° C. The solid was filtered and dried under vacuum.

1.-19. (canceled)

20. A crystalline form L2 of daclatasvir dihydrochloride wherein the diffraction peaks at 8.27, 9.54, 11.06, 13.61, 15.34, 19.01, 22.26, 23.24, 24.73, 25.35±0.2 degree two theta in an X-ray diffraction pattern.

21. The crystalline form L2 of daclatasvir dihydrochloride of claim 20, having X-ray powder diffraction pattern as depicted in FIG. 2.

22. The crystalline form L2 of daclatasvir dihydrochloride of claim 20, having characteristic infrared absorption at 3591, 3420, 3217, 1724, 1626, 1509, 1449, 1271, 1230, 1194, 1033, 906, 818, 741±2 cm-1.

23. A process for the preparation of crystalline form L2 of daclatasvir dihydrochloride comprising the steps of:
a) dissolving daclatasvir dihydrochloride in the solvent,
b) stirring the mixture,
c) isolating crystalline form L2 of daclatasvir dihydrochloride.

24. The process according to claim 23, wherein solvent is polar solvent or non-polar solvent or mixture thereof.

25. The process according to claim 24, wherein polar solvent is water, alcohol, nitrile, ether, ester, ketone, dimethylformamide, dimethyl sulfoxide or mixtures thereof.

26. The process according to claim 25, wherein alcohol is methanol, ethanol, butanol, propanol; nitrile is acetonitrile, propionitrile, butyronitrile; ether is tetrahydrofuran, dioxane, dimethoxyethane; ester is ethyl acetate, ethyl acetoacetate, butyl acetate, propyl acetate; ketone is acetone, methyl ethyl ketone, methyl isobutyl ketone or mixtures thereof.

27. The process according to claim 24, wherein non-polar solvents is hydrocarbon or chlorinated solvent.

28. The process according to claim 27, wherein hydrocarbon solvent is hexane, cyclohexane, n-heptane, pentane, cyclopentane, toluene; chlorinated solvent is methylene chloride, ethylene chloride, chloroform, carbon tetrachloride or mixtures thereof.
